(19) United States
(12) Patent Application Publication
(10) Pub. No.: US 2008/0146819 A1
(43) Pub. Date: Jun. 19, 2008

(54) Process for Preparing Levetiracetam

(75) Inventors: Arun Kanti Mandal, Vadodara (IN); Satish Wasudeo Mahajan, Vadodara (IN); Madan Kumar Sharma, Vadodara (IN); Apurba Chetia, Vadodara (IN); Nitesh Dolatram Chauhan, Vadodara (IN)

Correspondence Address: HAMRE, SCHUMANN, MUELLER & LARSON, P.C., P.O. BOX 2902, MINNEAPOLIS, MN, 55402-0902, US

(73) Assignee: RUBAMIN LIMITED, Vadodara (IN)

(21) Appl. No.: 11/886,012

(22) PCT Filed: Jan. 20, 2006
(86) PCT No.: PCT/IN2006/000019

§ 371 Date: Sep. 10, 2007

(30) Foreign Application Priority Data
Mar. 10, 2005 (IN) 264/MUM/2005

(51) Int. Cl. C07D 207/04

(52) U.S. Cl. 548/550

(57) ABSTRACT

Process for the preparation of (S)-(−)-α-ethyl-2-oxo-1-pyrrolidineacetamide of Formula (I) by the steps of condensation of (S)-2-amino butanol of Formula (II) and 4-halobutryl chloride, where halo group can be chloro, bromo or iodo in solvents to form α-ethyl-2-oxo pyrrolidine ethanol of Formula (III); oxidation of (S)-α-ethyl-2-oxo pyrrolidine ethanol to yield (S)-α-ethyl-2-oxo pyrrolidine acetic acid having the formula (IV); esterification of (S)-α-ethyl-2-oxo pyrrolidine acetic acid (IV) with an alcohol to provide alkyl ester of Formula (V) wherein, R is 14 Carbon atom; ammonolysis of alkyl esters of formula (V) with ammonia to provide (S)-(−)—α-ethyl-2-oxo-1-pyrrolidine acetamide of formula (1).

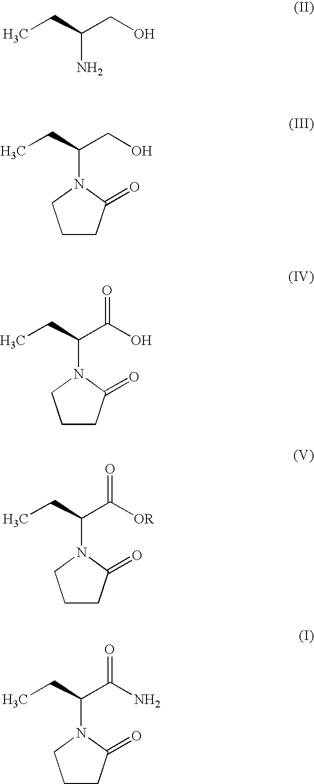

### FIELD OF INVENTION

The present invention relates to a novel process for preparing (S)-(−)-α-ethyl-2-oxo-1-pyrrolidine acetamide represented by formula (I)

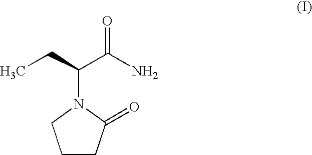

### BACKGROUND OF THE INVENTION

The compound of formula I is called Levetiracetam, which is useful as an agent for the treatment or prevention of epilepsy and other neurological disorders. British Pat. No. 1,309,692 teaches the compound (S)-(−)-α-ethyl-2-oxo-1-pyrrolidine acetamide of formula (I). The prior art methods for synthesis of Compound (I) could be summarised as follows:

U.S. Pat. No. 4,696,943 (Gobert et al.) describes the method either by reacting (S)-(−)-α-ethyl-2-oxo-1-pyrrolidineacetic acid successively with an alkyl chloro formate and with ammonia or by condensation followed by cyclization of 2-amino butanamide with 4-chlorobutyryl chloride. This process requires starting reactant with correct steriochemical configuration, the yields are often poor in the resolution.

GB 2225322 (Cossement E. et al.) claims (S)-(−)-α-ethyl-2-oxo-1-pyrrolidine acetamide preparation by hydrogenolysis of (S)-α-[2-(methylthio)ethyl]-2-oxo-1-pyriolidineacetamide in the presence of a desulfurising reagent. In this process the desulfurizing agents are not environment friendly.

U.S. Pat. No. 6,107,492 (Futagawa et al.) describes the method by optical resolution of racemic α—ethyl-2-oxo-1-pyrrolidineacetamide by means of preparative high performance liquid chromatography or continuous simulated moving bed chromatographic system using silicagel supported amylose tris (3,5-dimethylphenyl carbamate) as a packing material.

U.S. Pat. No. 6,124,473 (Cavoy et al.) claims an industrial scale enantiomeric resolution of racemic mixture of α-ethyl-2-oxo-1-pyrrolidineacetamide by simulated moving bed chromatography, using at least three columns filled with chiral stationary phase.

EP 1477478 (Surtees et al.) describes a process for preparing α-ethyl-2-oxo-1-pyrrolidineacetamide from lactam substituted 2-butenoic acid derivatives based on similar methodologies adopted by Boaz et al in U.S. Pat. No. 6,686,477 which involves preparation of enantiomerically pure lactum substituted propanoic acid derivatives by asymmetric hydrogenation of lactam substituted 2-propenoic acid derivatives. The dis advantage of the process is the reaction time necessary to obtain the conversion is very long and hence not attractive.

WO 03/014080 (Ates et al.) claims an improved process for (S)-(−)-α-ethyl-2-oxo-1-pyrrolidineacetamide from (S)-2-aminobutyric acid by alkylation of its methyl ester with ethyl-4-bromobutyrate, cyclization and amidation. Here again expensive optical active reactant is required.

WO 2004/069796 (Dolityzky) describes a process for preparing (S)-(−)-α-ethyl-2-oxo-1-pyrrolidineacetamide from (S)-2-aminobutyramide hydrochloride with 4-chlorobutyryl chloride in Acetonitrile or methyl tert butyl ether in the presence of a strong base. Here also expensive optical active reactant is required.

WO 2004/076416 (Surroca et al.) describes a method which comprises of preparation of aminomethyl derivatives of racemic α-ethyl-2-oxo-1-pyrrolidineacetamide, resolution followed by deaminomethylation of sufficiently pure enantiomeric intermediate to make (S)-(−)-α-ethyl-2-oxo-1-pyrrolidineacetamide. The loss during resolution makes this process unattractive

It is an object of the invention to provide a new cost effective chiral process for the preparation of (S)-(−)-α-ethyl-2-oxo-1-pyrrolidineacetamide, also known as Levetiracetam, in high yields.

It is a further object of the invention to provide a process for the preparation of (S)-(−)-α—ethyl-2-oxo-1-pyrrolidineacetamide without recourse to any chiral resolution step.

### SUMMARY OF THE INVENTION

Thus according to one aspect the present invention relates to a process for the preparation of (S)-(−)-o—ethyl-2-oxo-1-pyrrolidineacetamide of Formula (I), comprising the steps of:

i) condensation of (S)-2-amino butanol of Formula (II) and 4-halobutryl chloride, where halo group can be chloro, bromo or iodo in solvents to form α-ethyl-2-oxo pyrrolidine ethanol of Formula (III)

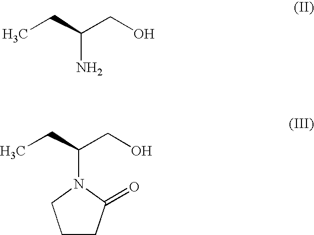

ii) oxidation of (S)-α-ethyl-2-oxo pyrrolidine ethanol to yield (S)-α-ethyl-2-oxo pyrrolidine acetic acid having the formula (IV)

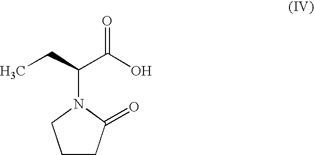

iii) esterification of (S)-α-ethyl-2-oxo pyrrolidine acetic acid (IV) with an alcohol to provide alkyl ester of Formula (V) wherein, R is 1-4 Carbon atom.

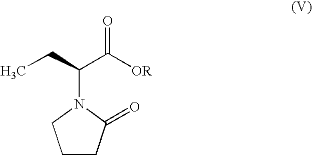

iv) ammonolysis of alkyl esters of formula (V) with ammonia to provide (S)-(−)—α-ethyl-2-oxo-1-pyrrolidine acetamide of formula (I).

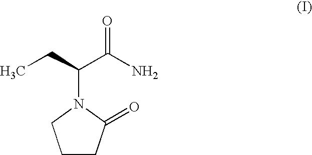

According to another aspect the invention relates to a process for the preparation of (S)-(−)-α—ethyl-2-oxo-1-pyrrolidineacetamide of Formula (I), comprising the steps of: i) condensation of (S)-2-amino butanol of Formula (II) and 4-halobutryl chloride, where halo group can be chloro, bromo or iodo in solvents to form α-ethyl-2-oxo pyrrolidine ethanol of Formula (III)

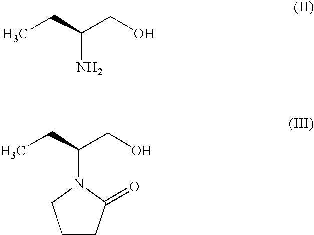

ii) oxidation of (S)-x-ethyl-2-oxo pyrrolidine ethanol to yield (S)-α-ethyl-2-oxo pyrrolidine acetic acid having the formula (IV)

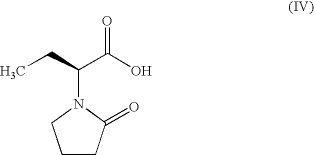

iii) reacting (S)-α-ethyl-2-oxo pyrrolidine acetic acid (IV) with alkyl haloformate of formula HalCOOZ in which Hal represents halogen atom and Z an alkyl radical having 1 to 4 Carbon atoms to provide alkyl ester of Formula (V) wherein, R is 1-4 Carbon atom.

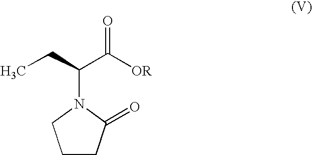

iv) ammonolysis of alkyl esters of formula (V) with ammonia to provide (S)-(−)-α-ethyl-2-oxo-1-pyrrolidine acetamide of formula (I).

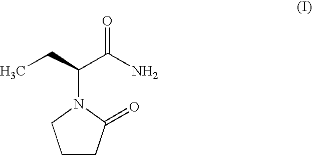

### DETAILED DESCRIPTION OF THE INVENTION

The enantiomerically pure S (+)-2-amino butanol represented by the general formula (II) is used as the starting material

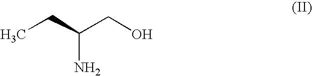

The new process of this invention comprises a sequential series of steps that involve:

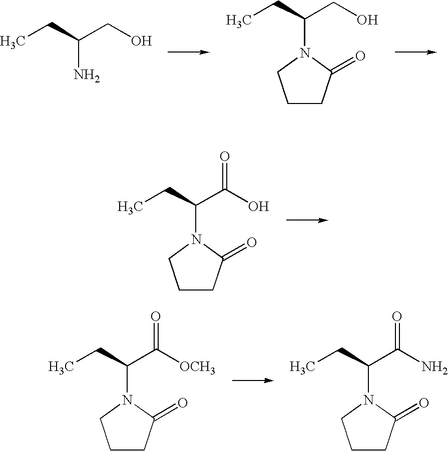

Condensation of (S)-2-amino butanol and 4-halobutryl chloride, where halo group can be chloro, bromo or iodo is carried out in the presence of an inorganic as well as tertiary organic base, in solvents, such as, benzene, toluene, xylene, trimethyl benzene, chlorinated solvents, eg, methylene chloride, chloroform, carbon tetrachloride, dichloroethane etc, Dimethyl formamide (DMF), Methyl t-butyl ether and Tetrahydrofuran, to make α-ethyl-2-oxo pyrrolidine ethanol of Formula (III). The temperature of the condentation is in the range of 0 to 40° C. and preferably 0 to 5° C.

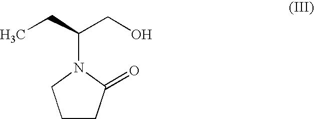

The step of oxidation of (S)-α-ethyl-2-oxo pyrrolidine ethanol is carried out in the presence of an oxidising agent in acidic, basic and neutral medium, preferably a basic medium, to yield (S)-α-ethyl-2-oxo pyrrolidine acetic acid having the formula (IV) in good yields at −10 to 50° C.

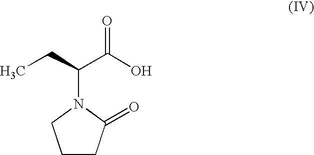

The oxidizing agent is selected from i) potassium permanganate in water (pH 7.0), in alkaline medium, pH (7-14) and even in acidic medium, pH (4-6), ii) Sodium or potassium dichromate in acidic medium.

The esterification of (S)-α-ethyl-2-oxo pyrrolidine acetic acid (IV) is effected with an alcohol in acidic medium or in presence of cationic ion exchange resin to make alkyl ester of Formula (V). Alternatively, the compound of Formula (V) can be formed by reacting the alkyl ester of Formula (IV) with alkyl haloformate of formula HalCOOZ in which Hal represents halogen atom and Z an alkyl radical having 1 to 4 Carbon atoms. The alkyl haloformate is preferably, commercially readily available, ethyl chloroformate, benzyl chloro formate and the like.

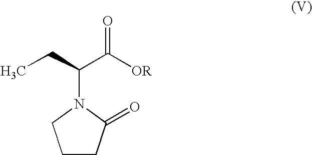

The ammonolysis of alkyl esters of formula (V) with ammonia in solvents is carried out in accordance with the method described by K. FOLKERS et al in J. Med. Chem., 14, 484-487 (1971) or under 3.5 to 4 kg of Ammmonia pressure in autoclave, yield (S)-(−)—α-ethyl-2-oxo-1-pyrrolidine acetamide of formula (I).

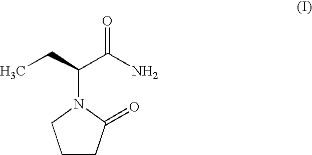

The present invention will now be described by the following nonlimiting examples.

### EXAMPLE-1

### Preparation of (S)-α-ethyl-2-oxo pyrrolidine ethanol (III)

184 g of anhydrous Na₂SO₄ is added to a suspension of 100 g (1.123 mole) of (S)-2-amino butanol in 800 ml of Toluene at ambient temperature. The mixture is cooled to 0 to 5° C. Then, 188 g of powder potassium hydroxide is added to the mixture followed by the addition of 173.4 g of 4-chlorobutyryl chloride in 100 ml of Toluene drop wise at 0° C., with vigorous stirring. Ten hrs later, the reaction mixture is filtered over Hyflo-cel and the filtrate evaporated under reduced pressure. The crude product is purified by high vacuum distillation (137-140° C. at 2 mm pressure).

Yield: 167.8 g (95%) [∝]²⁵=−27.46 (C=1, acetone)

### EXAMPLE-2

### Preparation of (S)-α-ethyl-2-oxo pyrrolidine acetic acid (IV)

A mixture of 225 g of (S)-α-ethyl-2-oxo pyrrolidine ethanol and a solution of 44.8 g of sodium carbonate in 4500 ml of water placed in a 10 litre round bottomed flask. Then 340 g of potassium permanganate is added to the reaction mixture with vigorous stirring, during 3-4 hours, cooling the mixture to 0°-5° C. by immersing in a bath of ice water. Allow the reaction mixture to attain room temperature gradually. 15 hours later, filter off the precipitated manganese dioxide, concentrated the filtrate to about 1000 ml under reduced pressure and acidified with dilute sulphuric acid up to pH 2 followed by the saturation with NaCl. Cover the solution with a layer of dichloromethane. Separate the dichloromethane layer and extract the aqueous layer two to three times with 100 ml portions of dichloromethane and distilled off on rotavapor. Recrystallised the crude acid (209 g) from 210 ml of toluene; filter and wash with toluene.

Yield: 130 gm (54%); MP 124° C.; [∝]²⁵=−24.32 (c=1, acetone)

### EXAMPLE-3

### Preparation of (S)-α-ethyl-2-oxo pyrrolidine acetic acid methyl ester (V)

A mixture of 34 g (S)-α-ethyl-2-oxo pyrrolidine acetic acid and 100 ml of methanol was taken in a 250 ml of round bottom flask fitted with reflux condenser and added 3.4 g ion exchange resin 22511 to the reaction. Allowed to reflux for 12 hours. Filter the resin. The crude product in methanol was taken as such for ammonolysis.

### EXAMPLE-4

### Preparation of (S)-(−)-α-ethyl-2-oxo-1-pyrrolidineacetamide (I)

(S)-α-ethyl-2-oxo pyrrolidine acetic acid methyl ester in MeOH from Example 3 is placed in autoclave under 4 kg of ammonia pressure at 25° C. The reaction mixture was allowed to stir for about 16 hours. The solvent was distilled off under reduced pressure. The crude (S)-(−)-α-ethyl-2-oxo-1-pyrrolidineacetamide 30 g was recrystallised from 240 ml Acetone to get 20.4 g pure product.

Yield: 20.4 (60%); MP: 117° C.; [∝]²⁵=−91.75 (C=1, acetone)

1. A process for the preparation of (S)-(−)-α-ethyl-2-oxo-1-pyrrolidineacetamide of Formula (I), comprising the steps of:
i) condensation of (S)-2-amino butanol of Formula (II) and 4-halobutryl chloride, where halo group can be chloro, bromo or iodo in solvents to form α-ethyl-2-oxo pyrrolidine ethanol of Formula (III) 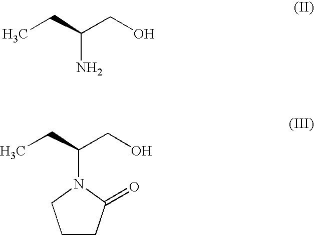
ii) oxidation of (S)-α-ethyl-2-oxo pyrrolidine ethanol to yield (S)-α-ethyl-2-oxo pyrrolidine acetic acid having the formula (IV) 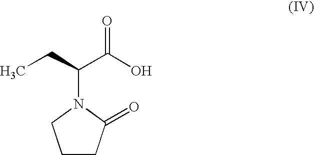
iii) esterification of (S)-α-ethyl-2-oxo pyrrolidine acetic acid (IV) with an alcohol to provide alkyl ester of Formula (V) wherein, R is 1-4 Carbon atom. 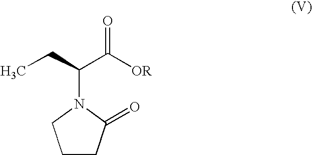
iv) ammonolysis of alkyl esters of formula (V) with ammonia to provide (S)-(−)-α-ethyl-2-oxo-1-pyrrolidine acetamide of formula (I).

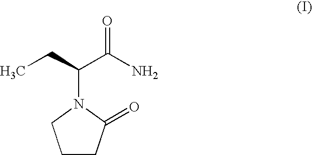

2. A process for the preparation of (S)-(−)-α-ethyl-2-oxo-1-pyrrolidineacetamide of Formula (I), comprising the steps of:
i) condensation of (S)-2-amino butanol of Formula (II) and 4-halobutryl chloride, where halo group can be chloro, bromo or iodo in solvents to form α-ethyl-2-oxo pyrrolidine ethanol of Formula (III) 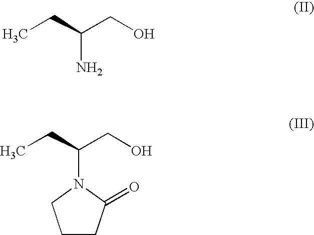
ii) oxidation of (S)-α-ethyl-2-oxo pyrrolidine ethanol to yield (S)-α-ethyl-2-oxo pyrrolidine acetic acid having the formula (IV) 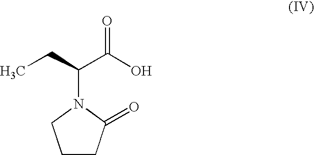
iii) reacting (S)-α-ethyl-2-oxo pyrrolidine acetic acid (IV) with alkyl haloformate of formula HalCOOZ in which Hal represents halogen atom and Z an alkyl radical having 1 to 4 Carbon atoms to provide alkyl ester of Formula (V) wherein, R is 1-4 Carbon atom. 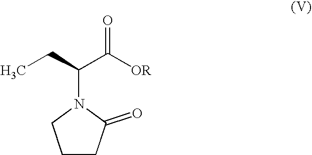
iv) ammonolysis of alkyl esters of formula (V) with ammonia to provide (S)-(−)-1-ethyl-2-oxo-1-pyrrolidine acetamide of formula (I).

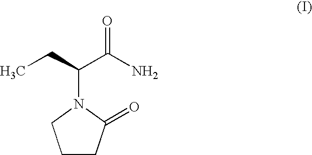

3. A process according to claim 1, wherein the condensation of (S)-2-amino butanol and 4-halobutryl chloride is carried out in the presence of an inorganic as well as tertiary organic base, in solvents, selected from benzene, toluene, xylene, trimethyl benzene, chlorinated solvents, dimethyl formamide (DMF), methyl t-butyl ether, tetrahydrofuran and the like.

4. A process according to claim 3, wherein the chlorinated solvent is selected from methylene chloride, chloroform, carbon tetrachloride, dichloroethane and the like.

5. A process according to any of claim 1, wherein the condensation is carried out at a temperature of between 0 and 40° C.

6. A process according to claim 1, wherein the condensation is carried out at a temperature of between 0 and 5° C.

7. A process according to claim 1 or 2, wherein the step of oxidation of (S)-α-ethyl-2-oxo pyrrolidine ethanol is carried out in the presence of an oxidising agent in acidic, basic or a neutral medium to yield (S)-α-ethyl-2-oxo pyrrolidine acetic acid having the formula (IV) in good yields at −10 to 50° C.

8. A process according to claim 7, wherein the step of oxidation is carried out in the presence of an oxidising agent in a basic medium.

9. A process according to claim 7, wherein the oxidizing agent is potassium permanganate in water (pH 7.0), in alkaline medium, pH (7-14) or in acidic medium, pH (4-6).

10. A process according to claim 7, wherein the oxidizing agent is sodium or potassium dichromate in acidic medium.

11. A process according to claim 1, wherein the esterification of (S)-α-ethyl-2-oxo pyrrolidine acetic acid (IV) is effected with an alcohol in acidic medium or in presence of cationic ion exchange resin to give alkyl ester of Formula (V).

12. A process for the preparation of (S)-(−)-α-ethyl-2-oxo-1-pyrrolidineacetamide of formula (I) comprising
i) reacting a mixture of (S)-x-ethyl-2-oxo pyrrolidine acetic acid and methanol in the presence of an ion exchange resin to form (S)-α-ethyl-2-oxo pyrrolidine acetic acid methyl ester of formula (V).
ii) subjecting the reaction mass to reflux followed by filtration;
iii) autoclaving (S)-α-ethyl-2-oxo pyrrolidine acetic acid methyl ester in MeOH with Ammonia and thereafter stirring the mixture;
iv) distilling of the solvent under reduced pressure to obtain crude (S)-(−)-α-ethyl-2-oxo-1-pyrrolidineacetamide; and
v) recrystalyzing the crude product to obtain substantially pure (S)-(−)-α-ethyl-2-oxo-1-pyrrolidineacetamide.
